(19) United States
(12) Patent Application Publication
(10) Pub. No.: US 2003/0003100 A1
(43) Pub. Date: Jan. 2, 2003

(54) DNA-antibody complexes to enhance gene transfer

(75) Inventors: Robert J. Levy, Merion Station, PA (US); Cunxian Song, Philadelphia, PA (US)

Correspondence Address: FOLEY AND LARDNER, SUITE 500, 3000 K STREET NW, WASHINGTON, DC, 20007, US

(73) Assignee: The Children's Hospital of Philadelphia

(21) Appl. No.: 10/143,858

(22) Filed: May 14, 2002

Related U.S. Application Data
(60) Provisional application No. 60/290,996, filed on May 16, 2001.

(51) Int. Cl. A61K 39/395; A61K 9/127

(52) U.S. Cl. 424/146.1; 424/450

(57) ABSTRACT

Transfection efficiency can be enhanced when a complex comprising a nucleic acid, an antibody that specifically binds the nucleic acid and a cationic macromolecule is introduced into mammalian cells. Delivery of nucleic acid into these cells enables transfection at levels comparable to conventional viral delivery.

### BACKGROUND OF THE INVENTION

1. Field of the Invention

The present invention relates to a gene therapy strategy, comprising an antibody-nucleic acid-cationic macromolecule complex, that enhances nucleic acid delivery to mammalian cells.

2. Description of the Related Art

Transfection is a common technique used for delivering nucleic acids to the interior of a cell. Transfection techniques known in the field include conventional mechanical procedures such as calcium phosphate precipitation, microinjection, electroporation, insertion of plasmid encapsulated in liposomes and viral vector delivery. These methods are not maximally effective and exhibit variable success in transfecting cells.

Delivery of naked DNA (DNA not complexed or covalently bound with a non-nucleic acid agent) may be the least efficient mode of gene transfer. Non-complexed DNA can be degraded by enzymes normally present in the cell or the extracellular environment and often does not remain localized. Moreover, in vitro plasmid DNA delivery with a transfection agent, such as a cationic lipid, improves nucleic acid transfer only slightly. In vivo plasmid DNA transfection is even less efficient. Typically, non-viral nucleic acid delivery strategies are less efficient than viral methods. Although viral vectors are generally more efficient systems of nucleic acid delivery, they can cause virus-mediated diseases, or symptoms thereof, in patients. For example, cell transduction with an adenovirus vector often induces an unwarranted inflammatory response.

Nucleic acid that comes into contact with a cell may or may not enter the interior of the cell or its nucleus. Mechanisms which further facilitate nucleic acid transport across membranes and nuclear localization, can increase transfection efficiency and are highly desireable. Studies have demonstrated that certain antibodies and fragments of antibodies are capable of crossing the cytoplasmic membrane, the nuclear membrane, or both, and of binding specifically with DNA in the cell. Madaio et al., 1998, J. Autoimmun. 11:535-538; van Helden et al., 1998, Biochim. Biophys. Acta 949:273-278; Yanase et al., 1997, J. Clin. Invest. 100:25-31. Accumulation of membrane-penetrating anti-DNA antibodies within the nucleus has been observed. One group attempted to use DNA-binding antibodies to facilitate transmembrane translocation of a nucleic acid, but was not able to reliably demonstrate gene transport into cells. Avrameas et al., 1998, Proc. Natl. Acad. Sci. U.S.A., 95:5601-5606. This group was able to achieve relatively efficient transmembrane transport of a plasmid by fusing a 19 residue polylysine polypeptide to the amino terminus of an antibody known to penetrate cells and bind DNA. However, transfection efficiency via this technique was equivalent to results seen using a standard transfection protocol with nucleic acid and a cationic macromolecule only.

### SUMMARY OF THE INVENTION

The inventors have discovered a nucleic acid delivery strategy that transfects cells at efficiencies comparable to those of traditional viral methods. The present invention encompasses a nucleic acid, an antibody that binds specifically with the nucleic acid, and a cationic macromolecule complexed with one or both of said nucleic acid and said antibody. The cationic macromolecule can have a targeting moiety covalently linked therewith, e.g., biotin or a polypeptide that specifically binds with a cell-surface protein. Preferably, the antibody comprises a nuclear targeting region and exhibits anti-nuclease I activity.

In a related vein, a method for delivering a nucleic acid to the interior of a cell, encompassing (A) exposing a cell to a complex comprised of (i) a nucleic acid, (ii) an antibody specifically bound with said nucleic acid and (iii) a cationic macromolecule non-covalently associated with one or both said nucleic acid and said antibody, is disclosed.

Further encompasing the invention is a method of making a transfection agent, comprising (A) incubating an antibody with a nucleic acid, (B) forming an antibody-nucleic acid complex and (C) adding a cationic macromolecule to said antibody-nucleic acid complex, is disclosed. Addition of the lipid reagent results in nanoparticle formation, which may facilitate cell entry.

In yet another aspect, the present invention describes a pharmaceutical composition comprising a nucleic acid, an antibody specifically bound with said nucleic acid, a cationic macromolecule non-covalently associated with one or both said nucleic acid and said antibody, and a pharmaceutically acceptable carrier in which said nucleic acid/antibody/cationic macromolecule are suspended.

### DETAILED DESCRIPTION OF PREFERRED EMBODIMENTS

With a goal of developing an effective means of delivering a nucleic acid to the interior of the cell, the inventors have discovered a non-viral strategy that improves cell transfection efficiency, without the adverse effects associated with viral mechanisms. Key to this strategy is a composition comprised of a nucleic acid, an antibody that binds specifically with the nucleic acid, and a cationic macromolecule complexed with one or both of the nucleic acid and the antibody.

Thus, the anti-nucleic acid antibody facilitates cellular uptake and increases nuclear localization, enhancing the biological effect of the nucleic acid on the cell (i.e., enhancing expression of an RNA or protein product encoded by the nucleic acid). A targeting polypeptide also can be linked covalently with the polycationic compound, in order to augment the specificity of nucleic acid delivery. The targeting polypeptide can be, for example, a portion of one of a ligand-receptor binding pair such as Fv, Fab′ and F(ab)′₂ fragment of an antibody that binds specifically with a cell surface protein.

Nucleic acid-delivery compositions of the present invention exhibit a superior capacity for delivering of the nucleic acid to desired cells or tissues. Moreover, nucleic acid delivery using the antibody/nucleic acid/cationic macromolecule complex can achieve transfection efficiencies comparable to that of viral vectors. Consequently, this transfection strategy can replace traditional viral methods of nucleic acid transfer and can be used in vivo, ex vivo and in vitro in experimental settings.

The antibody/nucleic acid/cationic macromolecule complex can be used in place of transfection compositions involving naked (linear or circular) nucleic acid vectors, nucleic acid-containing virus vectors, and nucleic acid vectors that are complexed with transfection enhancing agents, illustrated by polycationic agents such as polylysine. Thus, the composition described here can be used in place of substantially any prior art cell transfection composition.

As described in more detail below, transfection efficiency is markedly improved with the antibody/nucleic acid/cationic macromolecule complex. Nucleic acid delivery with an anti-nucleic acid antibody enhances cellular uptake and nuclear localization.

### The Nucleic Acid

The nucleic acid used in the composition described herein can be substantially any nucleic acid which one desires to transport to the interior of a cell or, in certain embodiments, to the nucleus of a cell.

The length of the nucleic acid is not critical to the invention. Any number of base pairs up to the full-length gene may be transfected. For example, the nucleic acid can be a linear or circular double-stranded DNA molecule having a length from about 100 to 10,000 base pairs in length, although both longer and shorter nucleic acids can be used.

The nucleic acid can be DNA or RNA, linear or circular and can be single- or double-stranded. DNA includes cDNA, triple helical, supercoiled, Z-DNA, and other unusual forms of DNA, polynucleotide analogs, antisense DNA, expression constructs comprising DNA encoding proteins such as a therapeutic proteins, transcribable constructs comprising DNA encoding ribozymes or antisense RNA, viral genome fragments such as viral DNA, plasmids, cosmids, DNA encoding a portion of the genome of an organism, gene fragments, and the like.

The nucleic acid can also be RNA. For example, antisense RNA, catalytic RNA, catalytic RNA/protein complex (i.e., a “ribozyme”), expression constructs comprising RNA that can be directly translated to generate a protein product, or that can be reverse transcribed and either transcribed or transcribed and translated to generate an RNA or protein product, respectively, transcribable constructs comprising RNA having any promoter/regulatory sequence necessary to enable generation of DNA by reverse transcription, a viral genome fragments such as viral RNA, RNA encoding a protein such as a therapeutic protein and the like. The nucleic acid can be selected on the basis of a known, anticipated, or expected biological activity that the nucleic acid will exhibit upon delivery to the interior of a target cell or its nucleus.

The nucleic acid can be prepared or isolated by any conventional means typically used to prepare or isolate nucleic acids. For example, DNA and RNA molecules can be chemically synthesized using commercially available reagents and synthesizers by methods that are described, for example, by Gait, 1985, in OLIGONUCLEOTIDE SYNTHESIS: A PRACTICAL APPROACH (IRL Press, Oxford). RNA molecules also can be produced in high yield via in vitro transcription methods using plasmids such as SP65, which is available from Promega Corporation (Madison, Wis.). The nucleic acid can be purified by any suitable means; many such means are known in the art. For example, the nucleic acid can be purified by reverse-phase or ion exchange HPLC, size exclusion chromatography, or gel electrophoresis. Of course, the skilled artisan will recognize that the method of purification will depend in part on the size of the DNA to be purified. The nucleic acid can also be prepared using any of the innumerable recombinant methods which are known or are hereafter developed.

Nucleic acids having modified internucleoside linkages can also be used in the compositions described herein. For example, nucleic acids containing modified internucleoside linkages which exhibit increased nuclease stability can be used. Such nucleic acids include, for example, those which contain one or more phosphonate, phosphorothioate, phosphorodithioate, phosphoramidate methoxyethyl phosphoramidate, formacetal, thioformacetal, diisopropylsilyl, acetamidate, carbamate, dimethylene-sulfide (—CH₂—S—CH₂—), dimethylene-sulfoxide (—CH₂—SO—CH₂—), dimethylene-sulfone (—CH₂—SO₂—CH₂—), 2′-O-alkyl, and 2′-deoxy-2′-fluoro-phosphorothioate internucleoside linkages.

The nucleic acid can be a therapeutic agent, such as an antisense DNA molecule that inhibits mRNA translation. Alternatively, the nucleic acid can encode a therapeutic agent, such as a transcription or translation product which, when expressed by a target cell to which the nucleic acid-containing composition is delivered, has a favorable therapeutic effect upon the cell. Examples of therapeutic transcription products include proteins (e.g., antibodies, enzymes, receptor-binding ligands, wound healing proteins, anti-restenotic proteins, anti-oncogenic proteins, and transcriptional or translational regulatory proteins), antisense RNA molecules, ribozymes, viral genome fragments, and the like. The nucleic acid can likewise encode a product useful as a marker for cells which have been transformed using the composition. Examples of markers include proteins having easily identifiable spectroscopic properties (e.g., green fluorescent protein; GFP) and proteins that are expressed on cell surfaces (i.e., which can be detected by contacting the target cell with an agent which specifically binds the protein).

By way of example, the nucleic acid can be selected from an expression construct encoding an anti-oncogenic protein and an anti-oncogenic antisense oligonucleotide. Examples of anti-oncogenic proteins include those encoded by the following genes: abl, akt2, apc, bcl2-alpha, bcl2-beta, bcl3, bcr, brcal, brca2, cbl, ccndl, cdk4, crk-II, csflr/fins, dbl, dcc, dpc4/smad4, e-cad, e2fl/rbap, egfr/erbb-l, elk], elk3, eph, erg, ets1, ets2, fer, fgr/src2, flil/ergb2, fos, fps/fes, fral, fra2, fyn, hck, hek, her2/erbb-2/neu, her3/erbb-3, her4/ erbb-4, hrasl, hst2, hstfl, ink4a, ink4b, int2/fgf3, jun, junb, jund, kip2, kit, kras2a, kras2b, ck, lyn, mas, max, mcc, met, mlhl, mos, msh2, msh3, msh6, myb, myba, mybb, myc, mycl1, mycn, nfl, nf2, nras, p53, pdgfb, pim1, pms1, pms2, ptc, pten, raft, rbl, rel, ret, ros1, ski, src1, tall, tglbr2, thral, thrb, tiam1, trk, vav, vhl, waf1, wnt1, wnt2, wt1, and yes1. Oligonucleotides which inhibit expression of one of these genes can be used as anti-oncogenic antisense oligonucleotides.

The nucleic acid described herein can be recombinantly engineered into a variety of known host vector systems that provide for replication of the nucleic acid on a large scale for the preparation of composition described herein. These vectors can be designed, using known methods, to contain the elements necessary for directing transcription, translation, or both, of the nucleic acid in a cell to which it is delivered. Methods which are known to the skilled artisan can be used to construct expression constructs having the protein coding sequence operably linked with appropriate transcriptional/translational control signals. These methods include in vitro recombinant DNA techniques and synthetic techniques. For example, see Sambrook et al., 1989, MOLECULAR CLONING: A LABORATORY MANUAL, Cold Spring Harbor Laboratory (New York); Ausubel et al., 1997, CURRENT PROTOCOLS IN MOLECULAR BIOLOGY, John Wiley & Sons (New York).

The nucleic acid encoding one or more proteins of interest can be operatively associated with a variety of different promoter/regulator sequences. The promoter/regulator sequences can include a constitutive or inducible promoter, and can be used under the appropriate conditions to direct high level or regulated expression of the gene of interest. Particular examples of promoter/regulatory regions that can be used include the cytomegalovirus promoter/regulatory region and the promoter/regulatory regions associated with the SV40 early genes or the SV40 late genes. Preferably, the human cytomegalovirus (hCMV) promoter is used in the present invention. However, substantially any promoter/regulatory region which directs high level or regulated expression of the gene of interest can be used.

It is also within the scope of the invention that the nucleic acid described herein contains a plurality of protein-coding regions, combined on a single genetic construct under control of one or more promoters. The two or more protein-coding regions can be under the transcriptional control of a single promoter, and the transcript of the nucleic acid can comprise one or more internal ribosome entry sites interposed between the protein-coding regions. Thus, an almost endless combination of different genes and genetic constructs can be employed.

### The Antibody

The antibody of the nucleic acid-containing composition can be a full-length (i.e., naturally occurring or formed by normal immunoglobulin gene fragment recombinatorial processes) immunoglobulin molecule (e.g., an IgG antibody) or an immunologically active (i.e., specifically binding) portion of an immunoglobulin molecule. The nucleic acid binding antibody binds at least one type of nucleic acid specifically. That is, the antibody is not simply one that binds, for example, any negatively charged polymer. The antibody can be one which binds only nucleic acids having a particular nucleotide sequence or one of a family of highly homologous sequences, or one which specifically binds the nucleic acid, regardless of its sequence. For example, the nucleic acid binding antibody can be one which binds substantially a nucleic acid of a particular type (e.g., double stranded DNA, single stranded DNA, single stranded RNA, or DNA-RNA hybrids) without regard to the sequence of the nucleic acid. By way of example, a nucleic acid-binding antibody exemplified herein is a murine monoclonal antibody that specifically binds single- or double-stranded DNA, without regard to sequence. Methods of generating and screening such antibodies are known and can be effected with standard experimentation.

Antibody fragments which recognize specific epitopes can be generated by known techniques. For example, such fragments include, but are not limited to: the F(ab)′₂ fragments which can be produced by pepsin digestion of the antibody molecule and the Fab′ fragments, which can be generated be generated by reducing disulfide bridges of the F(ab)′₂ fragments. Alternatively, Fab′ expression expression libraries can be constructed (Huse et al., 1989, Science, 246:1274-1281) to allow rapid and easy identification of monoclonal Fab′ fragments with the desired specificity. The nucleic acid-binding antibody used in the compositions described herein can be polyclonal or monoclonal antibody. Polyclonal antibodies are heterogeneous populations of antibody molecules derived from the sera of animals immunized with antigen, such as a target gene product, or an antigenic functional derivative thereof. Monoclonal antibodies are homogeneous populations of antibodies to a particular antigen and the antibody comprises only one type of antigen binding site to which the nucleic acid specifically binds. Preferably, the antibody is a full length antibody.

Also preferred, the antibody of the present invention comprises a nuclear targeting region, so that delivery to the nucleus of the nucleic acid in the composition described herein is enhanced. The presence of multiple positively charged amino acid residues in the complementarity determining regions (CDRs) of anti-DNA antibodies has been shown to enhance uptake of those antibodies into the nucleus. Madaio et al., 1998, J. Auto Immun. 11:535-538. The presence of nuclear localization-like motifs in one or more of the CDRs (preferably, CDR3) can also direct the antibody substance to the nucleus. Examples of nuclear localization motifs are reviewed by Hicks et al., 1995, Annu. Rev. Cell. Dev. Biol. 11:155-188.

Moreover, certain anti-DNA antibodies have been shown to inhibit endonuclease activity, such as DNase I activity. Yanase et al., 1997, J. Clin. Invest. 100:25-31. The antibody described herein inhibits endonuclease activity in the target cell or in the extracellular environment surrounding the target cell, thereby protecting the nucleic acid of the composition from degradation. This protection increases bioavailability and provides a longer period of time during which the nucleic acid/antibody substance/cationic macromolecule complex can enter the target or its nucleus.

### The Cationic Macromolecule

The cationic macromolecule is positively charged, comprises two or more art-recognized modular units (e.g., amino acid residues, fatty acid moieties, or polymer repeating units) and preferably is capable of forming supermolecular structures (e.g., aggregates, liposomes, or micelles) at high concentration in aqueous solution or suspension. Among the types of cationic macromolecules that can be used are cationic lipid and polycationic polypeptides and polymers.

Useful cationic lipids include commercially available cationic liposome compositions such as that marketed under the brand name LIPOFECTIN™. LIPOFECTIN™ is a mixture of the positively charged lipids N-[1-(2, 3-dioleyloxy) propyl]-N—N—N-trimethyl ammonia chloride (DOTMA) and dioleoyl phosphatidylethanolamine (DOPE). The identity of the cationic lipid is not critical; the positive charge and the ability to form micelles are believed to be important determinants of the suitability of the lipid. Substantially any cationic lipid (particularly including those known to be useful for complexing naked DNA in cell transfection methods) can be used in the compositions and methods described herein. Other commonly used cationic lipids include N-{1-(2,3-dioleoyloxy)propyl}-N,N,N-trimethylammonium methyl-sulfate (DOTAP), dioleoyl phosphatidylcholine (DOPC), dioctadecylamidoglycyl spermine (DOGS), DOTSA, and DOSPER.

Polycationic polypeptides include proteins having a relatively high net positive charge, and include, for example, homopolymers of amino acid residues that are positively charged under human physiological conditions. Examples of such homopolymers include polylysine, polyarginine, polyornithine, and polyhistidine. Homopolymers can comprise as few as several (e.g., 3-10) residues to several hundred or even several thousand residues. Polycationic polypeptides can also include polypeptides comprising amino acid residues that are positively charged under human physiological conditions, separated by non-charged or a relative small fraction (e.g., 50%, 25%, 10% or fewer) of negatively charged amino acid residues. Examples of polycationic proteins which can be used include naturally occurring proteins having a high net positive charge under human physiological conditions, such as myelin basic protein and various histones.

The cationic macromolecule can also be a polycationic polymer comprising repeating units having a moiety that is normally positively charged under human physiological conditions (i.e., wherein at least about 90% of the moiety exists in its positively-charged form at pH 7). Examples of such polymers include polybrene, and polyamines such as spermine, spermidine, prolamine, polyethylenimine, putrescine, cadaverine, and hexamine.

The cationic macromolecule can have a targeting moiety covalently linked therewith. The targetin moiety is preferably either the protein or the ligand of a protein-ligand pair, the protein and ligand exhibiting the property of binding with one another with high specificity. Examples of protein-ligand pairs are antibodies and their corresponding antigens, biotin and avidins such as streptavidin, and cell surface receptors that bind with specific proteins (e.g. fibroblast growth factors and their corresponding receptors). Substantially and known method of covalently linking a protein or ligand with the cationic macromolecule can be used. For example, a protein can be linked to a phospholipid such as that in the LIPOFECTIN™. product using a di-sulfhydryl compound such as N-succinimidyl-3-(2-pyridyldithio) propionate (SPDP).

### Making a Composition for Nucleic Acid Delivery

Detailed in this invention is a method for making a transfection agent, comprising (A) incubating an antibody with a nucleic acid, (B) forming an antibody-nucleic acid complex and (C) adding a cationic macromolecule to said antibody-nucleic acid complex. The critical steps are to first combine the nucleic acid with the anti-nucleic acid antibody in a ratio of 1 molecule nucleic acid to 10 molecules of antibody. Less efficient gene transfer may result if different nucleic acid:antibody proportions are used. After 3 hours of incubation time, the cationic macromolecule is added and nanoparticles are formed.

The amount of cationic macromolecule included in the composition can be determined based on either the amount of nucleic acid in the composition, or the combined amount of nucleic acid and antibody substance in the composition. For example, the molar ratio of cationic macromolecule to nucleic acid and antibody substance can be about 1:10. Preferably, the nucleic acid, the antibody substance and the cationic macromolecule are combined in a range of ratios (which can be determined experimentally) such that microparticulate complexes are formed. The complexes have a maximum dimension no greater than about 500 nm, but preferably not greater than 300 nm, 200 nm, or less.

Furthermore, the antibody-nucleic acid-cationic lipid complex can be modified to have targeting capabilities. Modifying the dioleyl-phosphatidy ethanolamine in LIPOFECTIN, for example, can achieve this result. A targeting moiety can be attached to the amino end of the cationic macromolecule by activating the dioleyl-phosphatidyl ethanolamine with SPDP and combining it with a sulfhydryl-containing targeting polypeptide. Examples of such targeting ligands include, but are not limited to, virtually any cell surface receptor ligand, including those involving cytokines, hormones (both peptide and nonpeptide), lipoprotein, and apparent viral receptors, such as the coxsackie-adenovirus (CAR) receptor-ligand system in order to target specific receptors. More generally stated, any receptor ligand to a constituitively expressed receptor, or an inducible receptor, or a receptor expressed due to gene vector transfection or transduction, or a mutant receptor occurring either spontaneously or through planned mutagenesis, or tumor specific receptors, either mutant or trans-phenotypic. These categories cover hundreds of examples. Alternatively, the targeting protein could be an anti-receptor antibody to most receptors. Thus, the modified transfection agent can enhance transfection efficiency as well as target specific signaling proteins.

### Method of Nucleic Acid Delivery

Another aspect of the present invention is a method for delivering a nucleic acid to the interior of a cell, comprising (A) exposing a cell to a complex comprised of (i) a nucleic acid, (ii) an antibody specifically bound with said nucleic acid and (iii) a cationic macromolecule non-covalently associated with one or both said nucleic acid and said antibody. The amount of transfection agent which should be used can be calculated based on the nucleic acid content of the complex. The capacity of the medium comprising or containing the transfection agent can also affect the amount of transfection agent to be used.

Once the composition described herein has been prepared, it can be used as a transfection agent in vivo, in vitro, or ex vivo, to enhance administration of nucleic acid to the interior of a cell. The identity of the cell is not critical, although it can be preferable to remove or degrade any cell wall that may be present prior to transfection.

### Proposed Theory of Operation

Formation of microparticulate compositions normally requires input of a great deal of energy, ordinarily provided in the form of rapid stirring, high pressure extrusion through restricted openings, or the like. It is unusual, therefore, that microparticulate complex formation occurs in the absence of high energy input.

Without being bound by any particular theory of operation, the inventors believe that microparticulate complex formation occurs by condensation of the nucleic acid. Condensation of the nucleic acid is enhanced by neutralization of the normally negatively charged nucleic acid by the positively charged moieties of the cationic macromolecule. The antibody substance is believed to act as a scaffold or template, upon which folding of charge-neutralized nucleic acid-cationic macromolecule complex can occur. When the cationic macromolecule has a substantially hydrophobic region (e.g., the fatty acyl moieties of a cationic lipid), association of the hydrophobic regions of nucleic acid-complexed macromolecule can drive further condensation of nuclei acid. The enthalpic energy gain attributable to association of the hydrophobic regions in a non-aqueous environment may provide the energy required to overcome the entropic burden of ordered microparticle formation. Of course, use of microparticulate nucleic acid-antibody-cationic macromolecule complexes are preferred, regardless of the manner in which such complexes are formed.

Addition of the cationic macromolecule to the nucleic acid-DNA complex results in the formation of nanoparticles after minimal vortexing. The particles can have a maximum dimension (e.g., the diameter for a spherical particle or the length of an elliptical particle, measured along its axis) in the range of 10 to 1000 nanometers, preferably about 500 nm (i.e., 500±50 nm). It is believed that nanoparticle formation is likely due to the strong hydrophobic interactions between the lipid reagent of the cationic macromolecule and the antibody-nucleic acid complex, in addition to the tight charge related binding of the cationic macromolecule to the nucleic acid. The nanoparticles are presumably taken up by cells by means of phagocytosis. Moreover, nuclear entry is facilitated by the antibody component of the complex and if the antibody exhibits anti-Nuclease I activity, then that can further enhance nucleic acid delivery.

Nanoparticulate complex formation may be responsible for some of the enhancement of nucleic acid uptake into the cells. In addition to reducing the physical size of the nucleic acid, complex formation may render the nucleic acid more amenable to binding with a portion of the cell membrane and passage therethrough. For example, if the antibody comprises an Fc portion, then that portion can bind Fc receptor proteins on the cell surface. This increases the association of the complex with a cell, thereby enhancing uptake of the complex. It may be that binding between an Fc receptor and an antibody triggers or enhances a cellular Fc receptor uptake/invagination method, thereby enhancing nucleic acid uptake. Association between a nucleic acid and a cationic macromolecule having a hydrophobic region can also render the nucleic acid more amenable to passage through a bilayer. For example, binding of the positively charged moiety of a cationic lipid with a negatively charged moiety of a nucleic acid can impart a more hydrophobic character to the nucleic acid. The more hydrophobic nucleic acid can thus translocate more easily across a lipid bilayer, either alone or when complexed with a cell surface receptor (e.g., an Fc receptor).

### The Pharmaceutically Acceptable Carrier

In yet another aspect of the present invention is a composition comprising a nucleic acid, an antibody specifically bound with said nucleic acid, a cationic macromolecule non-covalently associated with one or both said nucleic acid and said antibody, and a pharmaceutically acceptable carrier in which said nucleic acid/antibody/cationic macromolecule are suspended. Such a pharmaceutical composition can consist of the composition alone, in a form suitable for administration to a subject, or can comprise one or more pharmaceutically acceptable carriers, one or more additional ingredients, or some combination of these.

For oral administration, the pharmaceutical compositions can take the form of, for example, tablets or capsules prepared by conventional means with pharmaceutically acceptable excipients such as binding agents (e.g., gelatin, acacia, pregelatinized maize starch, polyvinylpyrrolidone and hydroxypropyl methylcellulose); fillers (calcium carbonate, sodium carbonate, lactose, microcrystalline cellulose, calcium phosphate, calcium hydrogen phosphate and sodium phosphate); lubricants (e.g., magnesium stearate, stearic acid, silica and talc); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The compounds can be formulated for parenteral administration (i.e., intravenous or intramuscular) by injection, via, for example, bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain g formulatory agents such as suspending, stabilizing and/or dispersing agents. Alternatively, the active ingredient can be in powder form for constitution with a suitable vehicle, e.g., sterile pyrogen-free water, before use. It is preferred that the TH cell subpopulation cells be introduced into patients via intravenous administration.

The compounds can also be formulated in rectal compositions such as suppositories or retention enemas, e.g., containing conventional suppository bases such as cocoa butter or other glycerides.

Douche preparations or suspensions for vaginal irrigation can be made by combining the composition described herein, with a pharmaceutically acceptable liquid carrier. As is known in the art, douche preparations can be administered using, and can be packaged within, a delivery device adapted to the vaginal anatomy of the subject. Douche preparations can further comprise various additional ingredients, including antioxidants, antibiotics, antifungal agents, and preservatives.

Vaginal preparations of the composition described herein can also be used for administration in utero of the nucleic acid described herein to an ovum, embryo, fetus, or a neonate during birth. Such preparations are preferably placed in the uterus of the woman bearing the ovum, embryo, fetus, or neonate, although such preparations can also be placed cervically or vaginally, or can be physically contacted with the embryo or fetus or on or within the chorionic or amniotic membranes.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions is contemplated include humans and other primates, mammals, including commercially relevant ones such as cattle, pigs, horses, sheep, cats and dogs, birds, fish and crustaceans.

The invention is now described with reference to the following examples, which are provided for illustration only. The invention is not limited to the examples, but rather includes all variations which are evident as a result of the teaching provided therein. These examples demonstrate that, by combining a cationic macromolecule with a DNA-antibody complex, transfection efficiency is comparable to conventional viral delivery strategies.

### EXAMPLE 1

### Preparation of an Antibody-DNA Conjugate and an Antibody-DNA-LIPOFECTIN™ Complex

40 μg of DNA (i.e., 2.9 μl of a 13.8 mg/ml DNA stock solution) encoding green fluorescent protein (GFP; Clontech, Palo Alto, Calif.) suspended in phosphate buffered saline (PBS) was diluted in 100 μl of Dulbecco's phosphate buffered saline (DPBS). 50 μl of the diluted suspension was placed into each of two tubes (i.e., 50 μl each), and the tubes were labeled “A” and “B”.

A preparation of mouse monoclonal anti-bovine DNA IgM (U.S. Biological; Swampscott, Mass.; recognizing double and single stranded DNA) was concentrated to 1.0 mg/ml using a Savant SPEEDVAC™ vacuum condensation system. Twenty microliters of the concentrated antibody preparationn was added to the tube labeled “A”, and the tube was mixed gently and incubated at 37 C. for 1 hour.

The solution in each of the tubes “A” and “B” was divided equally into two tubes and these tubes were designated A1, A2, B1 and B2. 5 μl LIPOFECTIN™ was added to tubes A1 and B1. All four tubes were incubated at room temperature for 35 minutes. A 0.9 ml aliquot of pre-warmed (37 C.) M199 medium was added to each of the four tubes and the contents of each tube were mixed. Table 1 indicates selected components in each tube.

TABLE 1

| | GFP- | Anti-DNA | LIPOFECTIN ™ | Total Volume in |
|---|---|---|---|---|
| Tube | DNA (μg) | Antibody (μg) | (μl) | PBS (μl) |
| A1 | 10 | 10 | 5 | 100 |
| A2 | 10 | 10 | 0 | 100 |
| B1 | 10 | 0 | 5 | 100 |
| B2 | 10 | 0 | 0 | 100 |

### EXAMPLE 2

### Transfection of A10 Smooth Muscle Cells In Vitro

Smooth muscle cells (A10) were used to test plasmid GFP-DNA transfection. Cells were transfected with anti-DNA antibody, GFP-DNA and LIPOFECTIN™; anti-DNA antibody and GFP-DNA; DNA and LIPOFECTIN™; and DNA only. 1×10⁵ cells in M199 medium, supplemented with 5% (v/v) fetal bovine serum (FBS), 100 Units/ml penicillin, and 100 μg per ml streptomycin (1% penn/strep) were added to each well of a 6-well cell culture plate. The cells were incubated at 37 C. for 18 hours prior to introduction of DNA. The cells were rinsed once with M199 medium which did not contain FBS and penn/strep, and then incubated for another 1 hour in M199 medium not containing FBS and penn/strep.

The medium was removed from the cells, and transfer the entire contents of one of the tubes A1, A2, B1 and B2 to each well. The cells were incubated at 37 C. for 2.5 hours, and then 3 μl of warmed (37 C.) FBS was added to each well. The cells were thereafter incubated at 37 C. for an additional 24 hours. Following the incubation, the transfection mixture was replaced with M199 medium supplemented with 2% (v/v) FBS and 1% (w/v) penn/strep. The cells were incubated at 37 C. for 24-48 hours in this medium, and fixed with 4% (v/v) paraformaldehyde. The cells were then mounted on a coverslip using VECTASHIELD™ mounting medium (Vector Laboratories, Inc., Burlingame, Calif.) and in the presence of 4′,6-diamidino-2-phenylindole (DAPI) in order to stain cell nuclei.

The cells were observed using a FITC-filtered fluoroscope for detection of GFP, and a DAPI filter for determining total cell numbers. The percentage of cells transfected was determined using NIH cell counting software. Table 2 indicates the percentage of cells transfected. Efficiency of transfection with the “three component complex” is enhanced 5 times compared with non-complexed DNA.

TABLE 2

| Tube | Percentage of cells transfected | Standard Deviation |
|---|---|---|
| A1 | 50.8 | ±6.0 |
| A2 | 3.4 | ±0.8 |
| B1 | 10.2 | ±3.6 |
| B2 | 1.0 | ±0.6 |

### EXAMPLE 3

### Cell Transfection With Rhodamine-Labeled DNA

The internalization and localization of anti-DNA antibody-DNA complex was assessed in cells using Rhodamine labeled DNA. DNA encoding P-galactosidase (plasmid PNGVLI-nt BetaGal, 7528bp; National Gene Vector Laboratory, Ann Arbor, Mich.) was labeled with Rhodamine using LABEL IT™ Labeling Kits, according to manufacturer's protocol. The labeled DNA was suspended in PBS to a final concentration of 0.1 μg/μl in PBS. 25 μl (2.5 μg) of this was conjugated to 2.5 μg of anti-DNA antibody as described in Example 1. 5 μl of LIPOFECTIN™ reagent was then added to form a complex of antibody/DNA/LIPOFECTIN™ before transfecting cells. Cells were transfected as described in Example 2. Localization of DNA was observed using a Texas Red filter on the fluoroscope.

Rhodamine labeled DNA was detected at the cell surface 4 hours after transfection. At 20 hours post transfection, the Rhodamine labeled DNA was seen in the cell, surrounding the nuclei. 28 hours post transfection, some Rhodamine labeled DNA entered the nuclei. At 40 hours, more Rhodamine labeled DNA was detected in the nucleus. This data demonstrates the time dependence of the transfection process.

### EXAMPLE 4

### In Vivo Transfection of the Antibody-DNA Complex

A cross-linking agent, N-succinimidyl-3-(2-pyridyldithio) propionate (SPDP), was used to immobilize mouse monoclonal anti-bovine DNA IgM (U.S. Biological, Swampscott, Mass.) onto a 1 dm square collagen film, consisting of approximately 1 mg of collagen on a polyurethane backing. 100 μg of GFP-DNA was bound to the immobilized antibody, therby immobilizing the DNA onto the collagen film. For the in vivo study.the plasmid GFP DNA tethered collagen film was incubated in a Lipofectin™-PBS solution(1:10, v/v) at room temperature for 35 minutes before implantation. Replication-defective adenovirus encoding GFP was similarly tethered to another piece of collagen film, using a mouse monoclonal IgG anti-knob antibodies (Selective Genetics, San Diego, Calif.), which had been immobilized onto the film. One collagen film was sewn onto the right atrial epicardial surface of pigs and results were examined after one week.

Extensive sub-epicardial gene expression of GFP was observed in both adenovirus transfected pigs and antibody-DNA-LIPOFECTIN™ transfected pigs. Pigs in which antibody-DNA was implanted exhibited greater GFP expression than pigs implanted with adenovirus-DNA. Furthermore, the inflammation typically associated with adenovirus-mediated transfections did not occur in pigs receiving the antibody-DNA implant. These data suggest that the antibody-DNA transfection method disclosed herein is more efficient and less harmful than traditional virus-mediated transfection methods, and can be practically useful in vivo, for example, in a pharmaceutical composition.

### EXAMPLE 5

### Nanoparticle Formation

A complex was formed, as described herein, among Rhodamine-labeled DNA, an anti-DNA antibody and LIPOFECTIN™. Corresponding complexes were made which lacked either the antibody or LIPOFECTIN™(DNA+antibody, DNA+LIPOFECTIN™ and DNA+antibody+LIPOFECTIN™). Each of these complexes was observed at 200× magnification using fluorescence microscopy. Nanoparticles having a maximum dimension of about 511±22 nanometers. These results indicate that the nucleic acid/antibody/cationic macromolecule complex described herein forms nanoparticles which can be taken up by cells (e.g. by phagocytosis).

It will be appreciated by those skilled in the art that changes could be made to the embodiments described above without departing from the broad inventive concept thereof. It is understood, therefore, that this invention is not limited to the particular embodiments disclosed, but induces modifications within the spirit of the scope of the present invention as defined by the appended claims.

What is claimed is:

1. A composition comprising a nucleic acid, an antibody that binds specifically with the nucleic acid, and a cationic macromolecule complexed with one or both of said nucleic acid and said antibody.

2. The composition of claim 1, wherein said antibody is selected from the group consisting of a full-length antibody, and Fc antibody fragment, and Fab′ antibody fragment, an F(ab)′₂ antibody fragment and a single chain antibody.

3. The composition of claim 2, wherein said antibody is a full-length antibody.

4. The composition of claim 1, wherein said antibody comprises a nuclear targeting region.

5. The composition of claim 1, wherein said antibody exhibits anti-nuclease I activity.

6. The composition of claim 4, wherein said antibody substance further comprises anti-nuclease I activity.

7. The composition of claim 1, wherein said cationic macromolecule is selected from the group consisting of a cationic lipid, a polycationic polypeptide, and a polycationic polymer.

8. The composition of claim 7, wherein said cationic macromolecule is a cationic lipid.

9. The composition of claim 1, wherein said cationic macromolecule is modified with a targeting moiety.

10. The composition of claim 1, wherein said nucleic acid is selected from the group consisting of DNA and RNA.

11. The composition of claim 10, wherein said nucleic acid is DNA.

12. The composition of claim 11, wherein said nucleic acid encodes green fluorescent protein.

13. The composition of claim 1, wherein said nucleic acid comprises a coding region operably linked to a promoter/regulatory region.

14. The composition of claim 13, wherein said promoter/regulatory region is selected from the group consisting of the cytomegalovirus (CMV) promoter/regulatory region, the SV40 early promoter/regulatory region, and the SV40 late promoter/regulatory region.

15. The composition in claim 14, wherein said promoter/regulatory region is a human cytomegalovirus (hCMV) promoter.

16. A method for delivering a nucleic acid to the interior of a cell, comprising (A) exposing a cell to a complex comprised of (i) a nucleic acid, (ii) an antibody specifically bound with said nucleic acid and (iii) a cationic macromolecule non-covalently associated with one or both said nucleic acid and said antibody.

17. The method for delivering in claim 16, wherein said antibody is selected from the group consisting of a full-length antibody, and Fc antibody fragment, and Fab′ antibody fragment, an F(ab)′₂ antibody fragment and a single chain antibody.

18. The method for delivering in claim 17, wherein said antibody is a full-length antibody.

19. The method for delivering in claim 16, wherein said nucleic acid is selected from the group consisting of DNA and RNA.

20. The method for delivering in claim 19, wherein said nucleic acid is DNA.

21. The method for delivering in claim 16, wherein said cationic macromolecule is selected from the group consisting of a cationic lipid, a polycationic polypeptide, and a polycationic polymer.

22. The method for delivering in claim 21, wherein said cationic macromolecule is a mixture of cationic liposome.

23. A method of making a transfection agent, comprising (A) incubating an antibody with a nucleic acid, (B) forming an antibody-nucleic acid complex and (C) adding a cationic macromolecule to said antibody-nucleic acid complex.

24. The method of making in claim 23, wherein said antibody is selected from the group consisting of a full-length antibody, and Fc antibody fragment, and Fab′ antibody fragment, an F(ab)′₂ antibody fragment and a single chain antibody.

25. The method of making in claim 24, wherein said antibody is a full-length antibody.

26. The method of making in claim 23, wherein said nucleic acid is selected from the group consisting of DNA and RNA.

27. The method of making in claim 26, wherein said nucleic acid is DNA.

28. The method of making in claim 23, wherein said cationic macromolecule is selected from the group consisting of a cationic lipid, a polycationic polypeptide, and a polycationic polymer.

29. The method of making in claim 28, wherein said cationic macromolecule is a mixture of cationic liposome.

30. A pharmaceutical composition comprising a nucleic acid, an antibody specifically bound with said nucleic acid, a cationic macromolecule non-covalently associated with one or both said nucleic acid and said antibody, and a pharmaceutically acceptable carrier in which said nucleic acid/antibody/cationic macromolecule are suspended.
